SEQUENCE LISTING (14 sequences)

SEQ ID NO: 1 - DNA, 1493 bp, Artificial sequence
  Synthetic
atcaaacttgagagtttgatcctggctcagaacgaacgctggcggcatgcctaatacatgcaagtcgaacgagatcttcggatctagtggcgcacgggtgcgtaacgcgtgggaatctgcccttgggttcggaataacttctggaaacggaagctaataccggatgatgacgtaagtccaaagatttatcgcccaaggatgagcccgcgtaggattagctagttggtggggtaaaggctcaccaaggcgacgatccttagctggtctgagaggatgatcagccacactgggactgagacacggcccagactcctacgggaggcagcagtagggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttgttcggaattactgggcgtaaagcgcacgtaggcggctattcaagtcagaggtgaaagcccggggctcaaccccggaactgcctttgaaactagatagcttgaatccaggagaggtgagtggaattccgagtgtagaggtgaaattcgtagatattcggaagaacaccagtggcgaaggcggctcactggactggtattgacgctgaggtgcgaaagcgtggggagcaaacaggattagataccctggtagtccacgccgtaaacgatgataactagctgtcagggcacatggtgttttggtggcgcagctaacgcattaagttatccgcctggggagtacggtcgcaagattaaaactcaaaggaattgacgggggcctgcacaagcggtggagcatgtggtttaattcgaagcaacgcgcagaaccttaccaacgtttgacatccctatcgcggatcgtggagacactttccttcagttcggctggataggtgacaggtgctgcatggctgtcgtcagctcgtgtcgtgagatgttgggttaagtcccgcaacgagcgcaaccctcgcctttagttgccagcatttagttgggtactctaaaggaaccgccggtgataagccggaggaaggtggggatgacgtcaagtcctcatggcccttacgcgttgggctacacacgtgctacaatggcgactacagtgggcagccacctcgcgagagggagctaatctccaaaagtcgtctcagttcggatcgttctctgcaactcgagagcgtgaaggcggaatcgctagtaatcgcggatcagcatgccgcggtgaatacgttcccaggccttgtacacaccgcccgtcacaccatgggagttggattcactcgaaggcgttgagctaaccgtaaggaggcaggcgaccacagtgggtttagcgactggggtgaagtcgtaacaaggtagccgtaggggaacctgcggctggatcacctcctttcta

SEQ ID NO: 2 - DNA, 400 bp, Artificial sequence
  Synthetic
taatcgggctacctgggatcgacttgtcttttgtgacctgaacaagctgcaagccggcctttccttgctcaacgccaatccacgcaaaattagtaccgtggaccgaaatgcctgcccgctcgccttcacgtttgaactcggggcgcataagggttgtcgcagtaaacgaggttgcgggcagcttttgggagaggatagctccattctcatagagatcctgagatcctgtcaccgatttgagccgaagccacccgctttcgacggacatccagtctgctgaggggttgctaccaaattgccaggctaagcttatcctgtcagcgaaatcgtcgtcagacaccggcgccgctatcggctgcgctgcgactttaggtttttgatgccgaaaaacaggttgacc

SEQ ID NO: 3 - DNA, 401 bp, Artificial sequence
  Synthetic
cagcttacgcaattgcaggagattactggtccacattgcaatccatatctgacgatagctgtttccggcacgctggcggcaaagacattgaattttgctgtgaccgacatatccggcggccttccgcatgatcgtgttctttcgaccatcgattatctgcggcaccttcatcatgcccttcccaatcaggtgaaggtgcatgacaggcctgttgattttgtgtttcaggcagaagatgtacctgtgaatattccaagcgtatcatgggaaacccggagatcctgggaccaaatcatattggttccagatctctattatttcaccagtcagggatatgaagatgctttcattggcaccacgccatggaacatgcggcaaaacaagatcatatggcgcggatc

SEQ ID NO: 4 - DNA, 400 bp, Artificial sequence
  Synthetic
gcccggaataagtttcaagcgggatggagcgatcgtccggttgacactggataagcctgagcgcttctccgtccagtttgacaatgaccggctacacaatctccatattgtcgcgggcgccctggtacccgagcaatcccagtccgacggaattacctattatggacctgggcttcacatccccgccgacggaagcaatcggtttccggtgaaatcgggtgatcgtatctatctggcaggaggcgcagttctccagggctcgtttgccctggatcatgtcaaagacgtcaaaatctctggtcggggccttctctacaatcccggtagcgccatcgacctggacggggctagcggcgtcgatgttcgcgatctgatcatcgtcaatgacgatcgcagcgat

SEQ ID NO: 5 - DNA, 1528 bp, Artificial sequence
  Synthetic
tagaaaggaggtgatccagccgcaccttccggtacggctaccttgttacgacttagtcctaatcaccgatcccaccttcgacggctccctccaaaaggttgggccaccggcttcgggtgttaccgactttcatgacttgacgggcggtgtgtacaaggcccgggaacgtattcaccgtggcgttgctgatccacgattactagcgactccgacttcatgaggtcgagttgcagacctcaatccgaactgagaccggctttttgggattcgctccaccttacggtattgcagccctttgtaccggccattgtagcatgcgtgaagcccaagacataaggggcatgatgatttgacgtcatccccaccttcctccgagttgaccccggcagtctcctatgagttcccaccataacgtgctggcaacatagaacgagggttgcgctcgttgcgggacttaacccaacatctcacgacacgagctgacgacaaccatgcaccacctgtttacgagtgtccaaagagttgaccatttctggcccgttctcgtatatgtcaagccttggtaaggttcttcgcgttgcatcgaattaatccgcatgctccgccgcttgtgcgggcccccgtcaattcctttgagttttagccttgcggccgtactccccaggcggggaacttaatgcgttagctgcgacacggagaccgtggaatggtccccacatctagttcccaacgtttacggcgtggactaccagggtatctaatcctgttcgctccccacgctttcgctcctcagcgtcagttacggcccagagatctgccttcgccatcggtgttcctcctgatatctgcgcattccaccgctacaccaggaattccaatctcccctaccgcactctagtctgcccgtacccactgcaggctggaggttgagcctccagttttcacagcagacgcgacaaaccgcctacgagctctttacgcccaataattccggacaacgcttgcaccctacgtattaccgcggctgctggcacgtagttagccggtgctttttctgcaggtaccgtcactttcgcttcttccctactaaaagaggtttacaacccgaaggccgtcatccctcacgcggcgttgctgcatcaggcttgcgcccattgtgcaatattccccactgctgcctcccgtaggagtctgggccgtgtctcagtcccagtgtggccggtcaccctctcaggccggctacccgtcgtcgccttggtgagccattacctcaccaacaagctgataggccgcgagtccatccttgaccaaaaaatctttccaccccctaaccatgcggttgagggtcgtatccggtattagacgtcgtttccaacgcttatcccagagtcaagggcaggttactcacgtgttactcacccgttcgccactgatccacagagcaagctctgcttcaccgttcgacttgcatgtgttaagcacgccgccagcgttcgtcctgagccaggatcaaactctccgtaaatga

SEQ ID NO: 6 - DNA, 399 bp, Artificial sequence
  Synthetic
catcggacgaactacccgtaccgtggagaacgccgcaatagcgtcaagctgctccgacggtccaattgctggcaaaaaccagagcccctggacctcaaatgacagatcctccctcacgttgaatccgaggttacgggcgtactccaggaaagcgagtcgattactcggggccaggttggcggacggcaagtggacgactaactcgaacgtcgcatccgggccagatccggtaccggctttgactttctcctcaggctgaaacgacgtgatctcttcgatctcgcgaatttgggcgtaccgctcaggcaattcgctgggatctaccatcagatcgtgaagcgcctggaaggcttcaagcctgcccgctacaaagatctcactcgttccaaactctttgcc

SEQ ID NO: 7 - DNA, 400 bp, Artificial sequence
  Synthetic
gagaaggttcggtactgacaccctcgtaatagcgagggccatccgtgcttggcaatgaaccatctagcggctcgcacaggccatttcggatcgccgcctccaaactggcgagatcagtgagctcgatcgtcctagcgatctgggacgcaatcatcgtgcgagagagggcggacctgtcgtcgtgagaacgctcgacgttttcggccgccgcagtttgaaccgaagccgcgagatctcggccaaccagttgcaatactcctgatggagtagaagtgtgctttttcttcaagcgagtgaccgtttgtgcctttgccgcatcccatggcaacgcgagtacccagacattgccgcacagttctcgtactgcggcctccgaaagctcttcggcgacggatattag

SEQ ID NO: 8 - DNA, 400 bp, Artificial sequence
  Synthetic
actttggtcttccaggctgttcgggcgaaagctgggtaggcatggacgccacccaggcggtgaccgacaacgggaccggacaggcaatcattctggcgttggccgccagcgacgagacccacgagtcatggaagcggttcaacctcgacgcgcaactagtggtcacctacaactcctacccggccgaccccaccaacttgggcatgctgacacctccgcgcacctgcggcaccctcaacgatcccgcatacatcaacccgaccctgcccttcacgctcgcagccacaatcagtgaccccgacgccaccgggtacggagtggagggccgattccgcatcatgccctacaacaacggggggatcaacgcgatcccagccagcgccccagccggatatctttc

SEQ ID NO: 9 - DNA, 400 bp, Artificial sequence
  Synthetic
tactcacagagttgcgcgaatcgacatctgtcagagcgttagtcagcaggaatgcgattgcccacagtcgatgctcgtcagggacgttcgggtttgcgcttttcacatccagcaacgggctccctggaagaatgtagtggcgaccctgacagcgaataatgctctcttctattccttcggccgtagcctcaataccgacaggaacgcaaacatccgtcaaccgcccagctcgtaaccgttccgagaagtcgaatcggtccttcaacatgaccggcaggtccttcggtatcctgaccacagaccagccagacacgcctccatgcttcttccagagcgcacgaagaattcgcgcctcaccatctgtctcgaatctcaccgcgtcccattcttcagcaaacag

SEQ ID NO: 10 - DNA, 1493 bp, Artificial sequence
  Synthetic
atcaaacttgagagtttgatcctggctcagaacgaacgctggcggcatgcctaatacatgcaagtcgaacgakmwcttcggwkmtagtggcgcacgggtgcgtaacgcgtgggaatctgcccttgggttcggaataacttctggaaacggaagctaataccggatgatgacgtaagtccaaagatttatcgcccaaggatgagcccgcgtaggattagctagttggtggggtaaaggctcaccaaggcgacgatccttagctggtctgagaggatgatcagccacactgggactgagacacggcccagactcctacgggaggcagcagtagggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttgttcggaattactgggcgtaaagcgcacgtaggcggctattcaagtcagaggtgaaagcccggggctcaaccccggaactgcctttgaaactagatagcttgaatccaggagaggtgagtggaattccgagtgtagaggtgaaattcgtagatattcggaagaacaccagtggcgaaggcggctcactggactggtattgacgctgaggtgcgaaagcgtggggagcaaacaggattagataccctggtagtccacgccgtaaacgatgataactagctgtcagggcacatggtgttttggtggcgcagctaacgcattaagttatccgcctggggagtacggtcgcaagattaaaactcaaaggaattgacgggggcctgcacaagcggtggagcatgtggtttaattcgaagcaacgcgcagaaccttaccaacgtttgacatccctatcgcggatcgtggagacactttccttcagttcggctggataggtgacaggtgctgcatggctgtcgtcagctcgtgtcgtgagatgttgggttaagtcccgcaacgagcgcaaccctcgcctttagttgccagcatttagttgggtactctaaaggaaccgccggtgataagccggaggaaggtggggatgacgtcaagtcctcatggcccttacgcgttgggctacacacgtgctacaatggcgactacagtgggcagccacctcgcgagagggagctaatctccaaaagtcgtctcagttcggatcgttctctgcaactcgagagcgtgaaggcggaatcgctagtaatcgcggatcagcatgccgcggtgaatacgttcccaggccttgtacacaccgcccgtcacaccatgggagttggattcactcgaaggcgttgagctaaccgtaaggaggcaggcgaccacagtgggtttagcgactggggtgaagtcgtaacaaggtagccgtaggggaacctgcggctggatcacctcctttcta

SEQ ID NO: 11 - DNA, 400 bp, Artificial sequence
  Synthetic
cgaaagatttcaggctggtgaggtcggacatatcctcataagcaccggtcgtcacggcagagcgcggcgatgccagcacgcaggcagccgtgtagagattttcgagaacgagtttcttgcagagaatgtcgtatcgctcgagataggacgcgcccttgaactccttgaaaatcgggaaatgcagcgaggattcacgcttggcggcacggcgcgatttatcggcatcctcgaccatgacaagccagccgacgaagggacgcgctgcatccgcgccgaacgcgccttcacggtaagcggtccagaaatcatgcgccgtgccgatggcctcctcggcacgattattcgcgttgttaccgaacgagggaccgacgtggcttttcatttcgatggcggcaatgag

SEQ ID NO: 12 - DNA, 400 bp, Artificial sequence
  Synthetic
gcaggggtatatggagcaacgccgagagcttgggcgtctgatgcgccatccctctttcgctcacgaatttcggcggtgctgccgtatcccaaattgctccacatcgcgactggcttagtttcacccttcaaggtcagatcgctgataggctcaaaagcgtttacacggctacgaacccacctttcgcgcccgactgcttggtcgaatgttgcgccttctgatacgcgagacttaatcgtcgttactgaaacatcatactggatggagaggtctgacagttcataaagaacaccgtcgatgacgagccagcccggcttagtgcgaaagccaaggatgcaatctactgatagcttcatgccttggcggcgctgcaattcctctagaggaatgtcgagcatcg

SEQ ID NO: 13 - DNA, 401 bp, Artificial sequence
  Synthetic
tattggttgagggtgcccatgatgcgcgccggttcaaaaaattttttgacgatacatcatgctcaataattaattgctttggcaaagataatgttactggaacaatagaaaacgaacaaaattctgcaaatgacgatgtgattggctttgttgatgtagattttgatcggatcaccggaacccatgccgataatgatgacataattcattctgtacatcacgattttgatcttgatgtttgcctgtccgatgcgattgaacgttatttcatcgaggtttgcgacgagcgcaaggttgttgattttggtgggtgcaggccatgcgtaaccaatatattggaatcattaaagccgctgtcagcattgcgatacgcaaatcagcgccatcgcttagggtattct

SEQ ID NO: 14 - DNA, 399 bp, Artificial sequence
  Synthetic
ggtcgataccagacggttgaaagacgcaatgttctggtcctccttcgcattgacctgaatagcttcaagccctgcgcgattgccgggtttcaggaccagcagaccaccgggcatttcctccgtgtcgatatgctgggttgcgtgatggtgggtgtctgcaatcctcaagtcaatatgctcaagcaccgatggcacttcgtcacataccatgtgccagaacttcctcatggctgttggcaggcgctttagggtttcgtgcgtgacaaggaggatttcgccgtggtgcggatcagcgtcggccatatgctcgactacgcgggcagcgacgcgatcattgcgcttcttgccatacagggctgttacgttcgcgtctggagcgatgcgtttgatctgcttcac

(19) United States
(12) Patent Application Publication
(10) Pub. No.: US 2024/0284914 A1
(43) Pub. Date: Aug. 29, 2024

(54) SYNERGISTIC MICROBIAL STRAINS FOR INCREASING THE ACTIVITY OF NITROGEN-FIXING MICROORGANISMS

(71) Applicant: University of Washington, Seattle, WA (US)

(72) Inventors: Sharon L. DOTY, Seattle, WA (US); Andrew Winslow SHER, Seattle, WA (US)

(73) Assignee: University of Washington, Seattle, WA (US)

(21) Appl. No.: 18/573,205

(22) PCT Filed: Jun. 17, 2022
(86) PCT No.: PCT/US2022/034077

§ 371 Date: Dec. 21, 2023

Related U.S. Application Data
(60) Provisional application No. 63/213,517, filed on Jun. 22, 2021.

(51) Int. Cl. A01N 63/20; A01P 21/00; C12N 1/20; C12N 15/11

(52) U.S. Cl. CPC A01N 63/20; A01P 21/00; C12N 1/205; C12N 15/11

(57) ABSTRACT

Embodiments of the present disclosure provide methods and compositions for increasing the nitrogen (N) fixation of diazotrophs or acquisition of N for a plant in need thereof. Embodiments of the methods and compositions comprise at least one live endophyte strain, wherein the live endophyte strain is isolated from one or more plants grown in a nutrient-limited and/or water-stressed environment. In some embodiments, the endophyte strain can be administered to a plant, wherein the endophyte strain synergistically increases the nitrogen fixation of the diazotrophic strain associated with the plant. In other embodiments, the diazotrophic strain is not associated with a plant. Embodiments of the present disclosure have broad application to reduce fertilizer requirements, increase plant carbon sequestration, increase production of hydrogen gas for use as an energy source or in chemical industries and to increase growth of industrial microbial strains, reducing the need for ammonium or nitrates in fermenters.

### CROSS-REFERENCE(S) TO RELATED APPLICATION(S)

This application claims the benefit of U.S. Provisional Application No. 63/213,517, filed Jun. 22, 2021.

### STATEMENT REGARDING SEQUENCE LISTING

The sequence listing associated with this application is provided in text format in lieu of a paper copy and is hereby incorporated by reference into the specification. The name of the text file containing the sequence listing is 3915-P 118WO2UW_Seq_list_Final_20220616_ST25.txt. The text file is 13 KB; was created on Jun. 16, 2022; and is being submitted via EFS-Web with the filing of the specification.

### BACKGROUND

Nitrogen (N) fixation in nature is an exclusively bacterial process that provides the essential N required for life by converting N₂ gas from the air into usable metabolites. Nitrogen can be shuttled between members of microbial communities, but diazotrophs (N fixing bacteria) are also commonly found in soils and associated with plants. Some plants house N fixing bacteria in dedicated structures, termed nodules, but bacteria can also live within plant tissues without causing disease as endophytes. Endophytes provide fixed N to the plant likely in exchange for receiving plant-provided sugars and other specialized molecules.

The appropriate plant microbiome can therefore profoundly improve plant growth and health. In addition to N, endophytes can also provide phosphorous and have been shown to increase plant tolerance to abiotic and biotic stresses.

Only in the last few years has the idea of using diazotrophic endophytes to produce N become accepted (Sharon L. Doty, 2017. Chapter 2: Endophytic Nitrogen Fixation. Controversy and a Path Forward. In Functional Importance of the Plant Endophytic Microbiome: Implications for Agriculture, Forestry and Bioenergy. Sharon L. Doty, editor. Springer doi. 10.1007/978-3-319-65897-1). Now it is broadly recognized that many non-leguminous plant species harbor symbiotic N-fixing endophytes and that free-living diazotrophs are often present in soils. This has made tapping into the N fixing capabilities of diazotrophs an area of interest. Some agricultural companies have been developing diazotrophic bio-inoculants, however simply applying single diazotrophic strains has not led to the crop yield gains that were anticipated.

Through energy demanding chemical processes, man-made N fertilizer can also be produced. However, due to the high-energy input, this is expensive and costs are passed on to consumers or farmers. Chemical fertilizers also negatively impact the environment due to the use of fossil fuels in their production, soil bacteria converting excess fertilizer into nitrous oxide (a potent greenhouse gas), and by disturbing aquatic ecosystems by leaching into waterways. In tropical agriculture, this pollution places sensitive coral reef ecosystems at risk.

Accordingly, there remains a need to provide technologies to increase the amount of fixed N produced by microbes in order to inexpensively generate nitrogen products that are not toxic to the environment. The method should be widely applicable to improve nitrogen availability for a variety of plants in a range of environments as well as any industrial process requiring nitrogen. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, in one aspect of the invention, the disclosure provides for a method to synergistically increase nitrogen acquisition in a plant in need thereof. The method can comprise generating an inoculant for a field treatment of a plant in need thereof. The inoculant can comprise a solution comprising an effective amount of at least one live endophyte strain, wherein the live endophyte strain is isolated from one or more plants grown in a nutrient-limited and/or water-stressed environment. The method can further comprise applying the inoculant to a plant in need thereof, wherein the live endophyte strain contacts at least one diazotrophic strain associated with the plant causing the diazotrophic strain to fix nitrogen at a higher rate compared to the nitrogen fixation rate of the diazotrophic strain in the absence of the live endophyte strain.

In another aspect of the invention, the disclosure provides for a method to synergistically increase nitrogen fixation of at least one live diazotrophic strain. The method can comprise contacting at least one live diazotrophic strain with an effective quantity of a solution comprising an effective quantity of at least one live endophyte strain, wherein the live endophyte strain is isolated from one or more plants grown in a nutrient-limited and/or water-stressed environment; and wherein contacting the live diazotroph strain with the live endophyte strain causes the live diazotroph strain to fix nitrogen at a higher rate compared to the nitrogen fixation rate of the live diazotroph strain in the absence of the live endophyte strain.

In another aspect of the invention, the disclosure provides for an inoculant to synergistically increase nitrogen acquisition in a plant in need thereof. The inoculant can comprise an effective quantity of a solution derived from a lyophilized formulation comprising an effective quantity of at least one live isolated endophyte strain, wherein the live isolated endophyte strain is isolated from one or more plants grown in a nutrient-limited and/or water-stressed environment.

In some embodiments, the at least one live isolated endophyte strain comprises a 16S nucleic acid sequence as set forth in SEQ ID NOs: 1, 5, and 10. In some embodiments, the at least one live isolated endophyte strain comprises a 16S nucleic acid sequence as set forth in SEQ ID NO: 1. In some embodiments, the at least one live isolated endophyte strain comprises a 16S nucleic acid sequence as set forth in SEQ ID NO: 5. In some embodiments, the at least one live isolated endophyte strain comprises a 16S nucleic acid sequence as set forth in SEQ ID NO. 10.

In some embodiments, the at least one live isolated endophyte strain comprises one or more markers selected from a sequence as set forth in SEQ ID NOs: 2-4, 6-9, and 11-14. In some embodiments, the at least one live isolated endophyte strain comprises three markers selected from a sequence as set forth in SEQ ID NOs: 2-4. In other embodiments, the at least one live isolated endophyte strain comprises four markers selected from a sequence as set forth in SEQ ID NOs: 6-9. In still other embodiments, the at least one live isolated endophyte strain comprises four markers selected from a sequence as set forth in SEQ ID NOs: 11-14.

In some embodiments, the at least one live isolated endophyte strain is of the species Sphingobium. In other embodiments, the at least one live isolated endophyte strain is of the species Herbiconiux.

In some embodiments, the nutrient-limited and/or water-stressed environment is a primary substrate. In some embodiments, the primary substrate is cobble or sand. In other embodiments, the nutrient-limited and/or water-stressed environment is one of a lava field, a desert, an arid environment, a semi-arid environment, and/or a charred environment.

In some embodiments, the plant in need thereof is selected from the group of a crop plant, a bioenergy crop plant, a forestry tree, a horticultural plant, a spice or medicinal plant, and a turfgrass.

In some embodiments, the inoculant comprises a solution comprising an effective quantity of two or more live isolated endophyte strains.

In some embodiments, the effective quantity of at least one live isolated endophyte strain is a quantity that causes the diazotrophic strain associated with the plant to increase nitrogen fixation by at least 5% compared to the nitrogen fixation rate of the diazotrophic strain associated with the plant in the absence of the at least one live isolated endophyte strain.

In some embodiments, the inoculant can further comprise at least one live isolated diazotrophic strain.

In some embodiments, the ratio of the at least one live isolated synergistic endophyte strain to the at least one live isolated diazotrophic strain is 1+n:1, wherein n is an integer from 0 to 20. In other embodiments, the ratio of the at least one live isolated endophyte strain to the at least one live isolated diazotrophic strain is 1:1+n, wherein n is an integer from 0 to 20.

In some embodiments, the inoculant is administered to a plant in need thereof, and the at least one live isolated endophyte strain contacts at least one diazotrophic strain associated with the plant causing the diazotrophic strain associated with the plant to fix nitrogen at a higher rate compared to the nitrogen fixation rate of the diazotrophic strain associated with the plant in the absence of the at least one isolated endophyte strain.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1. Acetylene reduction assay of diluted cultures showing synergistic partners can increase the nitrogen fixing activity of a variety of diazotrophs. Though the effects varied by strain, all synergistic partners increased the activity of at least two nitrogen fixers. White bars denote nitrogen fixers tested alone, while striped bars denote a mixture with synergistic partners.

FIG. 2. Acetylene reduction assay of diluted suspensions showing that a mixture of synergistic strains increases the activity of a variety of diazotrophs. Synergy Mix was treated as a single suspension containing OD₆₀₀ 0.2 of each strain. White bars denote nitrogen fixers tested alone, while striped bars denote a mixture with synergistic strains.

FIGS. 3A through 3B. Acetylene reduction assay of diluted cultures in nitrogen free media (NFM) (A) or diluted suspensions in NFM (B). Both A and B show that with increased ratios of synergistic strains to diazotrophs, nitrogen fixation activity increases as well. White bars denote nitrogen fixers tested alone, while striped bars denote a mixture with synergistic strains.

FIG. 4. Acetylene reduction assay performed with a mixture of diazotrophs that was treated as one cell suspension, each nitrogen fixer at OD₆₀₀ 0.2, which was mixed with a variety of strains related to WW5. The results show that the synergistic activity seen in partner strains is not a common trait of bacteria generally.

### DETAILED DESCRIPTION

This disclosure is based on the surprising and novel discovery that synergistic plant-associated bacteria strains isolated from a nutrient-limited and/or water-stressed environment, that can include, but is not limited to, Hawaiian lava bed-colonizing plants or cobble-dominated riparian zones, can synergistically increase the nitrogen fixation of any diazotrophic strains, whether the diazotrophic strains are free living, associated with the plant, or whether the diazotrophic strains are added (e.g. as an inoculant to a plant or any other means well-known to one of ordinary skill in the art) as part of a combination with the endophyte strains. The disclosed endophyte strains are synergistic partners that when combined with diazotrophic strains produce a combined nitrogen fixation capability that is greater than the sum of their individual nitrogen fixation capabilities. As such, the combination of one or more live synergistic bacteria strains can be used as field treatments to increase nitrogen acquisition in plants. In some embodiments, one or more live endophyte strains are added to the soil surrounding plants, synergistically increasing the nitrogen fixation of the existing diazotrophic strains associated with the plant. In other embodiments, one or more live endophyte strains are added in combination with one or more live diazotrophic strains and this combination is added to the soil surrounding plants to synergistically increase the nitrogen fixation of the existing diazotrophic strains associated with the plant. In other embodiments, the combination (e.g., endophyte strains and diazotrophic strains) can be used as part of a seed treatment/coating, or other applications to optimize plant growth or seed development by means well-known to one of ordinary skill in the art.

As described in more detail in the Examples, these endophyte strains were isolated, characterized and formulated in a specific combination to prepare as a plant inoculant Using acetylene reduction assays, the endophyte strains demonstrated nitrogen fixation and synergistic effects when combined with diazotrophic strains and the effects observed with the diversity of diazotrophic strains would suggest to one skilled in the art that the disclosed endophyte strains can function as synergistic partners with any diazotrophic strain to synergistically increase its nitrogen fixation capability. As such, these data demonstrate the utility of using one or more live endophyte strains as a synergistic partner to increase nitrogen fixation in host plants with a reduced need for external chemical fertilizers, providing an environmentally friendly and economically sustainable alternative to chemical fertilizers.

In accordance with the foregoing, in one aspect of the invention, the disclosure provides for a method to synergistically increase nitrogen acquisition in a plant in need thereof. The method can comprise generating an inoculant for a field treatment of a plant in need thereof. The inoculant can comprise a solution comprising an effective amount of at least one live endophyte strain, wherein the live endophyte strain is isolated from one or more plants grown in a nutrient-limited and/or water-stressed environment. The method can further comprise applying the inoculant to a plant in need thereof, wherein the live endophyte strain contacts at least one diazotrophic strain associated with the plant causing the diazotrophic strain to fix nitrogen at a higher rate compared to the nitrogen fixation rate of the diazotrophic strain in the absence of the live endophyte strain.

In another aspect of the invention, the disclosure provides for a method to synergistically increase nitrogen fixation of at least one live diazotrophic strain. The method can comprise contacting at least one live diazotrophic strain with an effective quantity of a solution comprising an effective quantity of at least one live endophyte strain, wherein the live endophyte strain is isolated from one or more plants grown in a nutrient-limited and/or water-stressed environment; and wherein contacting the live diazotroph strain with the live endophyte strain causes the live diazotroph strain to fix nitrogen at a higher rate compared to the nitrogen fixation rate of the live diazotroph strain in the absence of the live endophyte strain.

In another aspect of the invention, the disclosure provides for an inoculant to synergistically increase nitrogen acquisition in a plant in need thereof. The inoculant can comprise an effective quantity of a solution derived from a lyophillized formulation comprising an effective quantity of at least one live isolated endophyte strain, wherein the live isolated endophyte strain is isolated from one or more plants grown in a nutrient-limited and/or water-stressed environment.

As used herein, “nitrogen fixation”, “nitrogen acquisition”, and other grammatical variations of these phrases describe the chemical process by which diatomic nitrogen is converted into a nitrogen-containing organic or inorganic molecule to provide nitrogen in a form capable of being used in metabolism by living organisms.

The one or more live isolated endophyte strains are optionally isolated from one genus of plant, two genera of plants, three genera of plants, four genera of plants, five genera of plants, six genera of plants, seven genera of plants, eight genera of plants, nine genera of plants, ten genera of plants, or more than ten genera of plants. In another embodiment, the live isolated endophyte strains are optionally isolated from one species of plant, two species of plants, three species of plants, four species of plants, five species of plants, six species of plants, seven species of plants, eight species of plants, nine species of plants, ten species of plants, or more than ten species of plants.

The genera and species of plants from which the one or more live endophyte strains are isolated include, but are not limited to, plants which survive in nutrient-limited and/or water-stressed conditions. In some embodiments, nutrient-limited and/or water-stressed conditions include lava, sand, desert, rock, semi-arid and and climates, tropical, high pollution, high salinity, high minerality, charred, radiation-exposed, low oxygen, marine, and soil or regolith lacking any single necessary or preferred nutrient.

In some embodiments, the genera and species of plants from which the one or more live endophyte strains are isolated comprise a nutrient-limited and/or water-stressed environment that is a primary substrate. As used herein, the term “primary substrate” refers to the surface in which the plant grows is newly formed land. In some embodiments, the primary substrate is cobble or sand. In some embodiments, the primary substrate is lava. The lava can be a lava bed, la % a field, or lava plain. Additionally, the primary substrate can be in an environment of high pollution, high salinity, high minerality, charred, radiation-exposed, low oxygen, low water, marine, arid, semi-arid, or tropical. Typically, such primary substrates have a dearth of nutrients, and thus, plants that can establish initial growth are evolved to be able to compensate for such a lack of accessible nutrients. Such compensations can include the presence of a refined micro-biome that facilitates processing of nutrients, such as fixed nitrogen.

As used herein, the term “strain” refers to a genetic variant or subtype of a microorganism (e.g., bacterium).

The one or more live endophyte strains comprise bacteria isolated and selected from one genus of bacteria, two genera of bacteria, three genera of bacteria, four genera of bacteria, five genera of bacteria, six genera of bacteria, seven genera of bacteria, eight genera of bacteria, nine genera of bacteria, ten genera of bacteria, or more than ten genera of bacteria. In one embodiment, the plurality of live endophyte strains contains between six and eight genera of bacteria. The one or more live endophyte stains comprise bacteria isolated and selected from one species of bacteria, two species of bactena, three species of bacteria, four species of bacteria, five species of bacteria, six species of bacteria, or more than six species of bacteria. In one embodiment, the plurality of live endophyte strains is isolated and selected from between one and six species of a specified genus.

In still other embodiments, the one or more live endophyte strains are a helper strain that synergistically increases the nitrogen fixing rate of any diazotrophic strain. As used herein, “synergistically”, “synergistic”, or any grammatical variation of these words refer to an interaction between the at least one live endophyte strain (i.e., helper strain) and any dia.otrophic strain that produces a combined increase in nitrogen fixation greater than the sum of their individual effects (i.e., nitrogen fixing endophyte) or to a greater effect compared to the nitrogen fixation rate of the diazotrophic strain in the absence of the endophyte strain (i.e., non-nitrogen fixing endophyte).

In some embodiments, the one or more live endophyte strains comprise a 16S rRNA sequence as set forth in SEQ ID NOs. 1, 5, and 10. In some embodiments, the one or more live endophyte strains comprise a 16S rRNA sequence as set forth in SEQ ID NO: 1. In some embodiments, the live endophyte strains comprises a 16S rRNA sequence as set forth in SEQ ID NO. 5. In some embodiments, the live endophyte bacteria comprises a 16S rRNA sequence as set forth in SEQ ID NO: 10.

In still other embodiments, the one or more live endophyte strains comprise at least one marker comprising a sequence as set forth in SEQ ID NOs 2-4, 6-9, and 11-14. In some embodiments, the live endophyte strain comprises all three markers selected from SEQ ID NOs: 2-4. In some embodiments, the live endophyte strain comprises at least two markers selected from SEQ ID NOs: 2-4. In still other embodiments, the live endophyte strain comprises at least one marker selected from SEQ ID NOs: 2-4. In still other embodiments, the live endophyte strain comprises all four markers selected from SEQ ID NOs: 6-9. In some embodiments, the live endophyte strain comprises at least three markers selected from SEQ ID NOs: 6-9. In some embodiments, the live endophyte strain comprises at least two markers selected from SEQ ID NOs: 6-9. In still other embodiments, the live endophyte strain comprises at least one marker selected from SEQ ID NOs: 6-9. In still other embodiments, the live endophyte strain comprises all four markers selected from SEQ ID NOs: 11-14. In some embodiments, the live endophyte strain comprises at least three markers selected from SEQ ID NOs. 11-14. In some embodiments, the live endophyte strain comprises at least two markers selected from SEQ ID NOs: 11-14. In still other embodiments, the live endophyte strain comprises at least one marker selected from SEQ ID NOs: 11-14.

In still other embodiments, the one or more live isolated endophyte strains comprise a strain from at least one Sphingobium species and at least one Herbiconiux species. In still other embodiments, the live isolated endophyte strain comprises a 16S rRNA sequence as set forth in SEQ ID NO: 1, comprises all three markers selected from selected from SEQ ID NOs: 2-4, and is from the species Sphingobiun (i.e., helper strain 1, WW5). In other embodiments, the live isolated endophyte strain comprises a 16S rRNA sequence as set forth in SEQ ID NO. 5, comprises all four markers selected from selected from SEQ ID NOs: 6-9, and is from the species Herbiconiux (i.e., helper strain 2, 11R-B) In still other embodiments, the live isolated endophyte strain comprises a 16S rRNA sequence as set forth in SEQ ID NO: 10, comprises all four markers selected from selected from SEQ ID NOs: 11-14, and is from the species Sphingobium (i.e., helper strain 3, HT1-2). In some embodiments, the one or more live isolated endophyte strains comprise at least one strain selected from WW5, 11R-B, and HT1-2. In other embodiments, the live isolated endophyte strain comprises at least two strains selected from WW5, 11R-B, and HT1-2. In still other embodiments, the live isolated endophyte strain comprises three strains selected from WW5, 11R-B, and HT1-2.

As used herein, the term “marker” refers to a nucleotide sequence unique to each endophyte strain. For example, the WW5 strain (SEQ ID NO: 1) comprises the marker contig_60_9 (SEQ ID NO: 2), the marker contig_68_34 (SEQ ID NO: 3), and the marker contig_89_19 (SEQ ID NO: 4). The 11R-B strain (SEQ ID NO: 5) comprises the marker contig_2_456500 (SEQ ID NO: 6), the marker contig_3_405000 (SEQ ID NO: 7), the marker contig_4_300500 (SEQ ID NO: 8), and the marker contig_S_325500 (SEQ ID NO: 9). The HT1-2 strain (SEQ ID NO. 10) comprises the marker contig_3_1377 (SEQ ID NO: 11), the marker contig_1_601 (SEQ ID NO: 12), the marker contig_5_262 (SEQ ID NO: 13), and the marker contig_1_592 (SEQ ID NO: 14).

In some embodiments, the at least one bacteria strain that fixes nitrogen is or comprises an endophyte strain. This can be the same strain as comprised in the one or more live endophyte strains isolated from one or more plants grown in a nutrient-limited and/or water-stressed environment. In other embodiments, the nitrogen fixing endophyte strain is a different strain from the one or more live endophyte strains isolated from one or more plants grown in a nutrient-limited and/or water-stressed environment. In other embodiments, the at least one bacteria strain that fixes nitrogen is or comprises a non-endophyte bacteria strain. In still other embodiments, the at least one bacteria strain that fixes nitrogen is a diazotrophic strain.

One of ordinary skill in the art would understand that nitrogen is a required macronutrient for all plants. Because of this, the microbial strains disclosed herein (e.g., one or more live endophyte strains) can provide for additional nitrogen in any plant through the synergistic activity with any diazotrophic strain. In some embodiments, the plants can include but are not limited to crop plants. In some embodiments, the crop plants can include but are not limited to maize, wheat, barley, rice, canola, potato, and soy. In still other embodiments, the crop plants can include but are not limited to fruit, nut and vegetable crops, that can include but are not limited to tomatoes, strawberries, bananas, kale, spinach, lettuce, squashes, celery, broccoli, citrus, almond, hazel nut, walnut, cherry, apple, pear, and peach trees. In some embodiments, the crop plants can include but are not limited to bioenergy crops. In some embodiments, the bioenergy crops can include but are not limited to poplar, eucalyptus, miscanthus, switchgrass, and willow.

In still other embodiments, the plants can include forestry trees. In some embodiments, the forestry trees can include but are not limited to Douglas-fir, western hemlock, western redcedar, lodgepole pine, ponderosa pine, oak, maple, ash, spruce, and redwood.

In still other embodiments, the plants can include horticultural plants. In some embodiments, the horticultural plants can include but are not limited to azalea, rhododendron, roses, and hydrangea.

In still other embodiments, the plants can include spice or medicinal plants. In some embodiments, the spice or medicinal plants can include but are not limited to ginseng, cumin, coriander, and turmeric.

In still other embodiments, the plants can include turfgrasses. In some embodiments, the turfgrasses can include but are not limited to Kentucky bluegrass, fescues, and perennial ryegrass.

In some embodiments, one or more microbial strains described herein, along with the disclosed synergistic strains, can be added directly to the soil to boost the activity of diastrophic strains. In other embodiments, one or more microbial strains described herein, along with the disclosed synergistic strains, can be added to directly to plants comprising at least one diazotrophic strain to boost the activity of diastrophic strains. The synergistic strains (e.g., endophyte strains) can be applied to plants in any number of ways well-known to one of ordinary skill in the art. For example, in some embodiments, the synergistic strains are applied to plants through a foliar spray, applied as a solution (e.g., inoculant) to plant cuttings with or without roots, or applied to tissue culture plants. In some embodiments, the synergistic strains can be added to furrows or in irrigation solutions for plant and/or crop irrigation. In still other embodiments, the synergistic strains can be added to the soil as a dried powder or any combination of ways well-known to one of ordinary skill in the art. Once the synergistic strains have been incorporated into the plants, cuttings of these plants can also continue to contain the synergistic strains, indefinitely propagating the plant-microbe partnership. Thus, any parts of a plant or planting media that contained the synergistic strains can be continued sources of the synergistic strains.

In some embodiments, the isolated endophyte strains can be lyophilized following the isolation process. In other embodiments the isolated diazotrophic strains can be lyophilized following the isolation process. In still other embodiments, one or more microbial strains disclosed herein can be lyophilized following the isolation process. Microbial strains (e.g., endophyte strains, diazotrophic strains and/or other disclosed microbial strains) can be lyophilized following any techniques well-known to one of ordinary skill in the art.

As used herein, the term “inoculate” and grammatical variants thereof, refer to contacting a plant with the inoculant composition. In some embodiments, the inoculate is applied by spraying, soaking, dusting, gassing, and other techniques known in the art. The inoculant composition can also be mixed into soil or other substrate in which plant seeds are planted (previously or subsequently). In some embodiments, the inoculant can comprise a solution comprising an effective quantity of at least one live endophyte strain. In some embodiments, the inoculant can comprise a solution comprising an effective quantity of at least two live endophyte strains. In still other embodiments, the inoculant can comprise a solution comprising an effective quantity of three or more live endophyte strains. In some embodiments, the live endophyte strain is a live isolated strain. As used herein, “isolated strain” refers to a strain that is 100% pure, the strain does not include any contaminating strains. For example, live isolated endophyte strain WW5 is 100% pure WW5 strain without any contaminating strains.

In some embodiments, the inoculant comprises a ratio of at least one live isolated endophyte strain to at least one live isolated diazotrophic strain. In some embodiments, the ratio of the at least one live isolated endophyte strain to the at least one live isolated diazotrophic strain can be 1+n:1, wherein n is an integer from 0 to 20. In some embodiments, the ratio of the at least one live isolated endophyte strain to the at least one live isolated diazotrophic strain can be 1:1, 21, 3:1, 4:1, 5:1, 6:1, 7:1, 8:1, 9:1, 10:1, 11:1, 12:1, 13:1, 14:1, 15:1, 16:1, 17:1, 18:1, 19:1 and 20:1. In other embodiments, the ratio of the at least one live isolated endophyte strain to the at least one live isolated diazotrophic strain can be 1:1+n, wherein n is an integer from 0 to 20. In some embodiments, the ratio of the at least one live isolated endophyte strain to the at least one live isolated diazotrophic strain can be 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 and 1:20. In still other embodiments, the ratio of two live isolated endophyte strains to the at least one live isolated diazotrophic strain can be 1+n:1+n:1, wherein n is an integer from 0 to 20. In some embodiments, n can be the same for the first endophyte strain and the second endophyte strain. For example, the ratio can be 1:1:1, 2:2:1, 3:3:1, 4:4:1, 5:5:1, 6:6:1, 7:7:1, 8:8:1, 9:9:1, 10:10:1, 11:11:1, 12:12:1, 13:13:1, 14:14:1, 15:15:1, 16:16:1, 17:17:1, 18:18:1, 19:19:1 and 20:20:1. In other embodiments, n can be different for the first endophyte strain and the second endophyte strain. For example, the ratio can be 1:2:1, 2:3:1, 3:4:1, 4:5:1, and any variation that can be determined by one of ordinary skill in the art.

In still other embodiments, the inoculant can further comprise a solution comprising at least one strain of a live diazotroph. In some embodiments, the inoculant can further comprise a solution comprising at least two, three, four, five, six, seven or more strains of a live diazotroph. In some embodiments, the live diazotrophic strain is a live isolated diazotrophic strain. In some embodiments, the diazotrophic strain is HT1-9, the species is Azorhizobium sp., and the phylogenetic group is Alphaproteobacteria. In some embodiments, the diazotrophic strain is SherDot2 (SD2), the species is Azospirillum sp., and the phylogenetic group is Alphaproteobacteria. In some embodiments, the diazotrophic strain is WP4-2-2, the species is Burkholderia sp., and the phylogenetic group is Betaproteobacteria. In some embodiments, the diazotrophic strain is WPB, the species is Burkholderia vietnamiensis, and the phylogenetic group is Betaproteobacteria. In some embodiments, the diazotrophic strain is WP5, the species is Rahnella aceris, and the phylogenetic group is Gammaproteobacteria. In some embodiments, the diazotrophic strain is R10, the species is Rahnella aceris, and the phylogenetic group Gammaproteobacteria. In still other embodiments, the diazotrophic strain is SherDot1 (SD1), the species is Azotobacter beijerinckii, and the phylogenetic group Gammaproteobacteria.

As used herein, “an effective quantity” and grammatical variants thereof refer to the quantity of at least one live endophyte strain that causes a diazotrophic strain, either isolated in a culture or associated with a plant, to increase nitrogen fixation by at least 5% compared to the nitrogen fixation rate of a diazotrophic strain, either isolated in a culture or associated with a plant, in the absence of the endophyte strain. The increase in nitrogen fixation of a selected nitrogen fixing strain can be improved by at least 5%. For example, the increase in nitrogen fixation can be at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, or at least more than 30%. In some embodiments, the increase in nitrogen fixation can be at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%. In some embodiments, the nitrogen fixation of a selected nitrogen fixing bacteria can be improved by more than about 2-fold, such as about 3-fold, about 4-fold, about 5-fold, about 6-fold, about 7-fold, about 8-fold, about 9-fold, about 10-fold, or more (e.g., over a 100% nitrogen fixation up to and beyond 1000% more nitrogen fixation).

Nitrogen fixation in a selected nitrogen fixing strain can be improved by between about 5% and 2000%, between about 10% and 1500%, between about 15% and 1000%, between about 15% and 800%, between about 20% and 800%, between about 25% and 800%, between about 30% and 750% In some embodiments, nitrogen fixation can be improved by between about 50% and 500%, between about 50% r and 400%, between about 50% and 200%, and between about 75% and 100%.

Unless specifically defined herein, all terms used herein have the same meaning as they would to one skilled in the art of the present disclosure. For convenience, certain terms employed in the specification, examples, and appended claims are provided here. The definitions are provided to aid in describing particular embodiments and are not intended to limit the claimed invention, as the scope of the invention is limited only by the claims.

The use of the term “or” in the claims and specification is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an open and inclusive sense as opposed to a closed, exclusive or exhaustive sense. For example, the term “comprising” can be read to indicate “including, but not limited to.” The term “consists essentially of” or grammatical variants thereof indicate that the recited subject matter can include additional elements not recited in the claim, but which do not materially affect the basic and novel characteristics of the claimed subject matter. Additionally, the words “herein,” “above,” and “below,” and words of similar import, when used in this application, shall refer to this application as a whole and not to any particular portions of the application. Words using the singular or plural number also include the plural and singular number, respectively. The word “about” indicates a number within range of minor variation above or below the stated reference number. For example. “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc. of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

This disclosure describes the isolation, purification, inoculant preparation, and demonstrated activity of a plurality of live endophyte strains isolated from plants for use in inoculating plants to provide nutrients to plants without the need for excess chemical fertilizers and for methods to synergistically increase nitrogen fixation in a plurality of diazotroph strains.

### Example 1

### Isolation of Synergistic Strains

Plants growing in nutrient poor or water limited environments were sampled and surface sterilized. Approximately 10 g of tissue was then ground using a mortar and pestle into 15 ml sterile nitrogen-limited combined carbon medium (NLCCM) broth. The resulting slurry was then centrifuged at low speed to settle plant debris. A serial dilution was then made from the supernatant in NLCCM. In order to select for diazotrophs, the dilutions were plated onto NLCCM, as well as nitrogen free, NFCCM, agar plates. Isolated colonies were then re-streaked onto fresh NLCCM or NFCCM agar plates. When isolated colonies from the streaked plates were then re-streaked onto mannitol glutamate Luria broth (MG/L), the rich media allowed for the growth and isolation of multiple non-diazaotrophic strains that had formed a community with a diazotroph within what appears as one colony on nitrogen limited or nitrogen free plates. These emergent strains on rich media were candidates for synergy and tested by acetylene reduction for their ability to increase nitrogen fixation in diazotrophs.

### Example 2

### Acetylene Reduction Assay

### Bacterial Diluted Suspensions:

Bacteria were grown at 30° C. on nutrient rich MG/L agar plates. Nitrogen fixing strains were also grown on nitrogen limited NLCCM agar plates. Bacteria were then suspended in liquid NLCCM, or when specified, suspended in nitrogen free media (NFM) (Doty, S. L., Oakley, B., Xin, G. et al. Diazotrophic endophytes of native black cottonwood and willow. Symbiosis 47, 23-33 (2009)). Preference was given to the cells of nitrogen fixers grown on NL-CCM. The cells of each strain were then diluted to an optical density at 600 nm (OD₆₀₀) of 0.4 unless noted in Figure legends.

### Bacterial Cultures:

Bacteria were grown at 30° C. on MG/L agar plates. Nitrogen fixing strains were also grown on NL-CCM agar plates. Isolated colonies were selected, giving preference to the cells of nitrogen fixers grown on NL-CCM, for growth in 50 ml of MG/L without mannitol at 30° C. for 36 hrs. Cultures were centrifuged at 5000×g and washed in NFM repeating this process twice. The cultures of each strain were diluted to OD₆₀₀ of 0.4 in NFM.

### Strain Ratios:

When comparing ratios, nitrogen fixers were diluted to OD₆₀₀ 0.5 while the synergistic strains were diluted to create a series of dilutions with OD₆₀₀ 0.05, 0.1, 0.5, 2.5, and 5.0 such that when mixed together a series of ratios of nitrogen fixer to synergistic partners is formed.

### Acetylene Reduction:

Acetylene reduction to ethylene was used as a proxy for nitrogen fixation activity because the nitrogenase enzyme performs both chemistries. Cell mixes were created by combining diluted suspensions or diluted cultures in equal parts using a total volume of 150 ul by adding them to 17 ml amber septa vials that had been prepared with 6 ml of NLCCM agar. The resulting 11 ml of headspace was then dosed with 0.1 ml of 98.6% acetylene. Following incubation at 30° C. for two days unless noted, headspace was sampled by removing 5 ml of air from 22 ml gas chromatography vials and replacing it with 5 ml of headspace from the experimental vials. Samples were analyzed by a gas chromatograph with a flame ionization detector (GC-FID, TRACE GC ULTRA, Thermo Scientific) using a HayeSep R column. High purity N2 (g) was used as the carrier, H2 (g) as the fuel, and synthetic air as the oxidizing gas. Peak area was then converted to parts per million (ppm) using a standard curve of ethylene concentrations.

### Example 3

### Sequencing of Synergistic Strains

Full genomic sequencing of the three synergistic strains indicates that all three strains are unique and novel species.

TABLE 1
Strains were identified using the Type (Strain) Genome Server
(TYGS) protocol. The TYGS database consists of >15,000 type-
strain species/sub-species genomes. A d₄ score ≤70.0%
is the threshold for a potentially novel species (1-2).

| | Taxonomic | | |
|---|---|---|---|
| Strain | Classification | d₄ (95% CI) | Closest Type Strain |
| WW5 | Sphingobium sp. | 66.3 (63.3-69.1) | S. yanoikuyae |
| | | | ATCC51230 |
| 11R-B1 | Herbiconiux sp. | 24.5 (22.2-27.0) | H. ginsengi |
| | | | CGMCC 4.3491 |
| HT1-2 | Shingobium sp. | 68.1 (65.1-71.0) | S. yanoikuyae |
| | | | ATCC51230 |

Strain-specific regions of the genomes for these three strains (e.g., WW5, 11R-B1, and HT1-2).

In addition to using the 16s ribosomal genes for strain identification (SEQ ID NOs: 1, 5, and 10), strain-specific primers for Sphingobium sp. WW5, Herbiconiux sp. 11R-B1, and Sphingobium sp. HT1-2 were designed using protocols adapted from Stets et. al. (Stets M I, et al., Quantification of Azospirillum brasilense FP2 bacteria in wheat roots by strain-specific quantitative PCR. Appl Environ Microbiol. 2015; 81(19):6700-6709. doi: 10.1128/AEM. 01351-15) and Jo et al. (Jo, J., et al., Microbial community analysis using high-throughput sequencing technology: a beginner's guide for microbiologists. J Microbiol. 58, 176-192 (2020)). For each strain, the FASTA genome sequences were partitioned into 500 bp non-overlapping segments using the shred.sh (v.2.3.7) program from BBMap (v38.96) Local databases were constructed from 7 complete genomes for each genera, Sphingobium and Herbiconiux, downloaded from the NCBI Reference Sequence database. The following steps were completed in Geneious Prime (v2022.1.1 Build 2022-03-15 11:43). The segmented FASTA files of the candidate sequences for each strain were subjected to BLASTn searches against the local databases and the segments for which there were no hits were retained. The filtered list of candidate sequences was then subjected to BLASTn searches against a second local database of complete genomes composed of the inventors' internal lab strains, again retaining only the segments for which there were no-hits. Finally, the remaining candidate sequences were submitted online as queries against the full NCBI nucleotide database, and the segments for which there were no matches were designated as the unique sequences and used to design the strain-specific primers.

A single primer set was designed for each unique sequence. The primers were designed in Geneious Prime, using the Primer3 plug-in (v2.3.7) with the following settings: i) optimal amplicon length 400 nt, range 300-500 nt, ii) primer length 22-25 nt, iii) Tm range 57-63 degC, max Tm difference between primers 2 degC, iv) and optimal % GC 50%, range 40%-60%. The resulting products (primer sets and amplicon sequences together) were mapped to the genome assemblies of their respective strain, and products that fell completely within a CDS were used as the candidate primer sets.

A total of 47 strain specific primer set (SSPs) were identified for WV5. Of those. 18 SSPs targeted coding sequences (CDS), 3 of which are in known genes. The primer sets and predicted products are included Table 2. A total of 29 strain specific primer set (SSPs) were identified for 11R-B. Of those, 10 SSPs targeted annotated coding sequences (CDS), only one of which targeted known genes, and the other 9 SSPs targeted CDS annotated as hypothetical proteins. Included in Table 2 are 4 primer sets and the predicted products for the single identified gene hit and 3 arbitrarily chosen primers sets that landed in hypothetical proteins. A total or 217 strain specific primer set (SSPs) were identified for HT1-2. Of those, 89 SSPs targeted annotanted coding sequences (CDS), 7 of which targeted known genes, and the other 82 SSPs targeted CDS annotated as hypothetical proteins. Included in Table 2 are primer sets and the predicted products for 2 SSPs that target identified genes, and 2 that target hypothetical proteins.

TABLE 2
Candidate primer sets with amplicon sequences

Contig_60_9_PCR_Product Sphingobium sp. WW5
TAATCGGGCTACCTGGGATCGACTTGTCTTTTGTGACCTGAACAAGCTGCAAGCCGGCCTTTCCTTGCTCA
ACGCCAATCCACGCAAAATTAGTACCGTGGACCGAAATGCCTGCCCGCTCGCCTTCACGTTTGAACTCGG
GGCGCATAAGGGTTGTCGCAGTAAACGAGGTTGCGGGCAGCTTTTGGGAGAGGATAGCTCCATTCTCATA
GAGATCCTGAGATCCTGTCACCGATTTGAGCCGAAGCCACCCGCTTTCGACGGACATCCAGTCTGCTGAG
GGGTTGCTACCAAATTGCCAGGCTAAGCTTATCCTGTCAGCGAAATCGTCGTCAGACACCGGCGCCGCTA
TCGGCTGCGCTGCGACTTTAGGTTTTTGATGCCGAAAAACAGGTTGACC
(SEQ ID NO: 2)
Contig_68_34_PCR_Product Sphingobium sp. WW5
CAGCTTACGCAATTGCAGGAGATTACTGGTCCACATTGCAATCCATATCTGACGATAGCTGTTTCCGGC
ACGCTGGCGGCAAAGACATTGAATTTTGCTGTGACCGACATATCCGGCGGCCTTCCGCATGATCGTGTT
CTTTCGACCATCGATTATCTGCGGCACCTTCATCATGCCCTTCCCAATCAGGTGAAGGTGCATGACAGG
CCTGTTGATTTTGTGTTTCAGGCAGAAGATGTACCTGTGAATATTCCAAGCGTATCATGGGAAACCCGG
AGATCCTGGGACCAAATCATATTGGTTCCAGATCTCTATTATTTCACCAGTCAGGGATATGAAGATGCT
TTCATTGGCACCACGCCATGGAACATGCGGCAAAACAAGATCATATGGCGCGGATC
(SEQ ID NO: 3)
Contig_89_18_PCR_Product Sphingobium sp. WW5
GCCCGGAATAAGTTTCAAGCGGGATGGAGCGATCGTCCGGTTGACACTGGATAAGCCTGAGCGCTTCTCC
GTCCAGTTTGACAATGACCGGCTACACAATCTCCATATTGTCGCGGGCGCCCTGGTACCCGAGCAATCCC
AGTCCGACGGAATTACCTATTATGGACCTGGGCTTCACATCCCCGCCGACGGAAGCAATCGGTTTCCGGT
GAAATCGGGTGATCGTATCTATCTGGCAGGAGGCGCAGTTCTCCAGGGCTCGTTTGCCCTGGATCATGTC
AAAGACGTCAAAATCTCTGGTCGGGGCCTTCTCTACAATCCCGGTAGCGCCATCGACCTGGACGGGGCTA
GCGGCGTCGATGTTCGCGATCTGATCATCGTCAATGACGATCGCAGCGAT
(SEQ ID NO: 4)
Contig_2_456500-456999_PCR_Product 11RB
CATCGGACGAACTACCCGTACCGTGGAGAACGCCGCAATAGCGTCAAGCTGCTCCGACGGTCCAATTGC
TGGCAAAAACCAGAGCCCCTGGACCTCAAATGACAGATCCTCCCTCACGTTGAATCCGAGGTTACGGGC
GTACTCCAGGAAAGCGAGTCGATTACTCGGGGCCAGGTTGGCGGACGGCAAGTGGACGACTAACTCGAA
CGTCGCATCCGGGCCAGATCCGGTACCGGCTTTGACTTTCTCCTCAGGCTGAAACGACGTGATCTCTTC
GATCTCGCGAATTTGGGCGTACCGCTCAGGCAATTCGCTGGGATCTACCATCAGATCGTGAAGCGCCTG
GAAGGCTTCAAGCCTGCCCGCTACAAAGATCTCACTCGTTCCAAACTCTTTGCC
(SEQ ID NO: 6)
Contig_3_405000-405499_PCR_Product 11RB
GAGAAGGTTCGGTACTGACACCCTCGTAATAGCGAGGGCCATCCGTGCTTGGCAATGAACCATCTAGCG
GCTCGCACAGGCCATTTCGGATCGCCGCCTCCAAACTGGCGAGATCAGTGAGCTCGATCGTCCTAGCGA
TCTGGGACGCAATCATCGTGCGAGAGAGGGGGACCTGTCGTCGTGAGAACGCTCGACGTTTTCGGCCG
CCGCAGTTTGAACCGAAGCCGCGAGATCTCGGCCAACCAGTTGCAATACTCCTGATGGAGTAGAAGTGT
GCTTTTTCTTCAAGCGAGTGACCGTTTGTGCCTTTGCCGCATCCCATGGCAACGCGAGTACCCAGACAT
TGCCGCACAGTTCTCGTACTGCGGCCTCCGAAAGCTCTTCGGCGACGGATATTAG
(SEQ ID NO: 7)
Contig_4_300500-300999_PCR_Product 11RB
ACTTTGGTCTTCCAGGCTGTTCGGGCGAAAGCTGGGTAGGCATGGACGCCACCCAGGCGGTGACCGAC
AACGGGACCGGACAGGCAATCATTCTGGCGTTGGCCGCCAGCGACGAGACCCACGAGTCATGGAAGCG
GTTCAACCTCGACGCGCAACTAGTGGTCACCTACAACTCCTACCCGGCCGACCCCACCAACTTGGGCA
TGCTGACACCTCCGCGCACCTGCGGCACCCTCAACGATCCCGCATACATCAACCCGACCCTGCCCTTC
ACGCTCGCAGCCACAATCAGTGACCCCGACGCCACCGGGTACGGAGTGGAGGGCCGATTCCGCATCAT
GCCCTACAACAACGGGGGGATCAACGCGATCCCAGCCAGCGCCCCAGCCGGATATCTTTC
(SEQ ID NO: 8)
Contig_5_325500-325999_PCR_Product 11RB
TACTCACAGAGTTGCGCGAATCGACATCTGTCAGAGCGTTAGTCAGCAGGAATGCGATTGCCCACAGTC
GATGCTCGTCAGGGACGTTCGGGTTTGCGCTTTTCACATCCAGCAACGGGCTCCCTGGAAGAATGTAGT
GGCGACCCTGACAGCGAATAATGCTCTCTTCTATTCCTTCGGCCGTAGCCTCAATACCGACAGGAACGC
AAACATCCGTCAACCGCCCAGCTCGTAACCGTTCCGAGAAGTCGAATCGGTCCTTCAACATGACCGGCA
GGTCCTTCGGTATCCTGACCACAGACCAGCCAGACACGCCTCCATGCTTCTTCCAGAGCGCACGAAGAA
TTCGCGCCTCACCATCTGTCTCGAATCTCACCGCGTCCCATTCTTCAGCAAACAG
(SEQ ID NO: 9)
Contig_3_1377_PCR_Product Sphingobium sp. HT1-2
CGAAAGATTTCAGGCTGGTGAGGTCGGACATATCCTCATAAGCACCGGTCGTCACGGCAGAGCGCGGCG
ATGCCAGCACGCAGGCAGCCGTGTAGAGATTTTCGAGAACGAGTTTCTTGCAGAGAATGTCGTATCGCT
CGAGATAGGACGCGCCCTTGAACTCCTTGAAAATCGGGAAATGCAGCGAGGATTCACGCTTGGCGGCAC
GGCGCGATTTATCGGCATCCTCGACCATGACAAGCCAGCCGACGAAGGGACGCGCTGCATCCGCGCCGA
ACGCGCCTTCACGGTAAGCGGTCCAGAAATCATGCGCCGTGCCGATGGCCTCCTCGGCACGATTATTCG
CGTTGTTACCGAACGAGGGACCGACGTGGCTTTTCATTTCGATGGCGGCAATGAG
(SEQ ID NO: 11)
Contig_1_601_PCR_Product Sphingobium sp. HT1-2
GCAGGGGTATATGGAGCAACGCCGAGAGCTTGGGCGTCTGATGCGCCATCCCTCTTTCGCTCACGAATT
TCGGCGGTGCTGCCGTATCCCAAATTGCTCCACATCGCGACTGGCTTAGTTTCACCCTTCAAGGTCAGA
TCGCTGATAGGCTCAAAAGCGTTTACACGGCTACGAACCCACCTTTCGCCCCCGACTGCTTGGTCGAAT
GTTGCGCCTTCTGATACGCGAGACTTAATCGTCGTTACTGAAACATCATACTGGATGGAGAGGTCTGAC
AGTTCATAAAGAACACCGTCGATGACGAGCCAGCCCGGCTTAGTGCGAAAGCCAAGGATGCAATCTACT
GATAGCTTCATGCCTTGGCGGCGCTGCAATTCCTCTAGAGGAATGTCGAGCATCG
(SEQ ID NO: 12)
Contig_5_262_PCR_Product Sphingobium sp. HT1-2
TATTGGTTGAGGGTGCCCATGATGCGCGCCGGTTCAAAAAATTTTTTGACGATACATCATGCTCAATAA
TTAATTGCTTTGGCAAAGATAATGTTACTGGAACAATAGAAAACGAACAAAATTCTGCAAATGACGATG
TGATTGGCTTTGTTGATGTAGATTTIGATCGGATCACCGGAACCCATGCCGATAATGATGACATAATTC
ATTCTGTACATCACGATTTTGATCTTGATGTTTGCCTGTCCGATGCGATTGAACGTTATTTCATCGAGG
TTTGCGACGAGCGCAAGGTTGTTGATTTTGGTGGGTGCAGGCCATGCGTAACCAATATATTCGAATCAT
TAAAGCCGCTGTCAGCATTGCGATACGCAAATCAGCGCCATCGCTTAGGGTATTCT
(SEQ ID NO: 13)
Contig_1_592_PCR_Product Sphingobium sp. HT1-2
GGTCGATACCAGACGCTTGAAAGACGCAATGTTCTGGTCCTCCTTCGCATTGACCTGAATAGCTTCAAGC
CCTGCGCGATTGCCGGGTTTCAGGACCAGCAGACCACCGGGCATTTCCTCCGTGTCGATATGCTGGGTTG
CGTGATGGTGGGTGTCTGCAATCCTCAAGTCAATATGCTCAAGCACCGATGGCACTTCGTCACATACCAT
GTGCCAGAACTTCCTCATGGCTGTTGGCAGGCGCTTTAGGGTTTCGTGCGTGACAAGGAGGATTTCGCCG
TGGTGCGGATCAGCGTCGGCCATATGCTCGACTACGCGGGCAGCGACGCGATCATTGCGCTTCTTGCCAT
ACAGGGCTGTTACGTTCGCGTCTGGAGCGATGCGTTTGATCTGCTTCAC
(SEQ ID NO: 14)

### Example 4

### Individual Synergistic Strains Induce Higher Activity in Diazotrophs

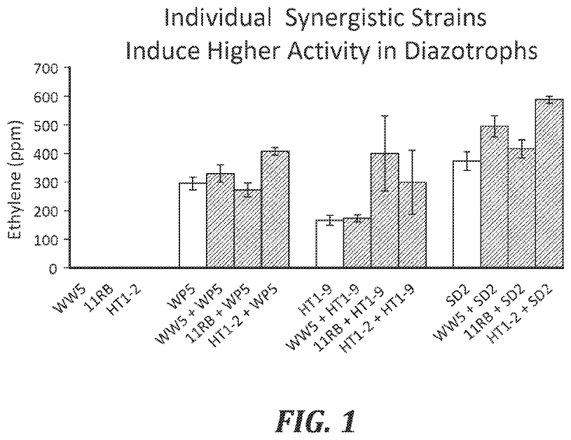

Acetylene reduction assay of diluted cultures showed synergistic partners can increase the nitrogen fixing activity of a variety of diazotrophs. FIG. 1. Though the effects varied by strain, all synergistic partners increased the activity of at least two nitrogen fixers. In FIG. 1, white bars represent nitrogen fixers tested alone (e.g., WP5, HT1-9, and SherDot2 (SD2)). As illustrated for the WP5 diazotrophic strain, the addition of synergistic partner strains (striped bars) WW5 and HT1-2 resulted in the largest synergistic increase in nitrogen fixation as represented by acetylene reduction to ethylene. Similar to the WP5 diazotrophic strain, the addition of the synergistic partner strains 11RB and HT1-2 resulted in the largest synergistic increase in nitrogen fixation. Similar results were observed for the SD2 diazotroph strain. However, all three synergistic partner strains (e.g., WW5, 1 RB, and HT1-2) synergistically increased the nitrogen fixation of the SD2 diazotroph strain.

### Synergy Mix Induces Higher Acetylene Reduction

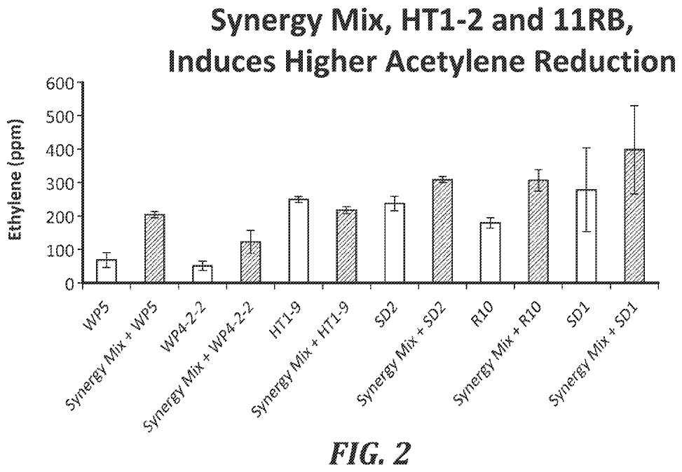

Acetylene reduction assay of diluted suspensions showed that a mixture of synergistic strains increases the activity of a variety of diazotrophs (white bars). Synergy mix was treated as a single suspension containing OD₆₀₀ 0.2 of each strain. As illustrated in FIG. 2, a synergy mix (striped bars), comprising synergy partner strains HT1-2 and 11RB increased nitrogen fixation as represented by acetylene reduction to ethylene. The increase in nitrogen fixation was observed in a variety of diazotrophic strains (white bars), including WP5, WP4-2-2, HT1-9, SD2, R10, and SD1.

The example diazotrophic strains exhibiting increased nitrogen fixation activity represent a diverse selection of diazotrophic species. Because the synergistic strains (e.g., NW5, 11RB, and HT1-2) increase nitrogen fixation in this diverse selection of diazotrophic strains, one skilled in the art would recognize that this result (i.e., synergistic strains increasing nitrogen fixation) is representative for all diazotrophic strains. As such, the results disclosed in this Example are not limited to the specific diazotrophic strains disclosed in embodiments of the claimed invention but can be applied to all diazotrophic strains.

TABLE 3
Diversity of diazotrophic strains

| Strain | Species | Phylogenetic group |
|---|---|---|
| HT1-9 | Azorhizobium sp. | Alphaproteobacteria |
| SherDot2 | Azospirillum sp. | Alphaproteobacteria |
| WP4-2-2 | Burkholderia sp. | Betaproteobacteria |
| WPB | Burkholderia vietnamiensis | Betaproteobacteria |
| WP5 | Rahnella aceris | Gammaproteobacteria |
| R10 | Rahnella aceris | Gammaproteobacteria |
| SherDot1 | Azotobacter beijerinckii | Gammaproteobacteria |

Synergistic Strains Induced More Activity with Increased Concentration after Incubation with Diazotroph

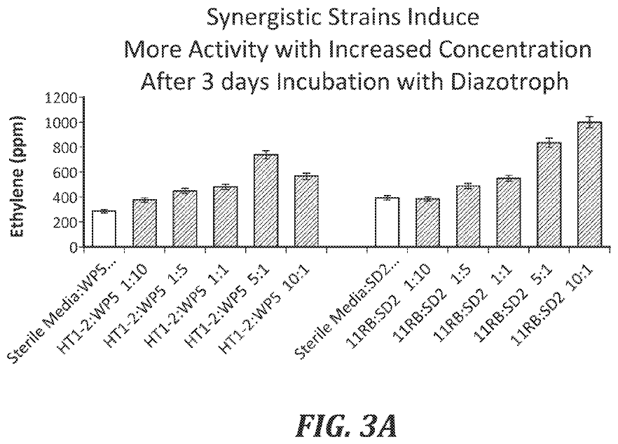

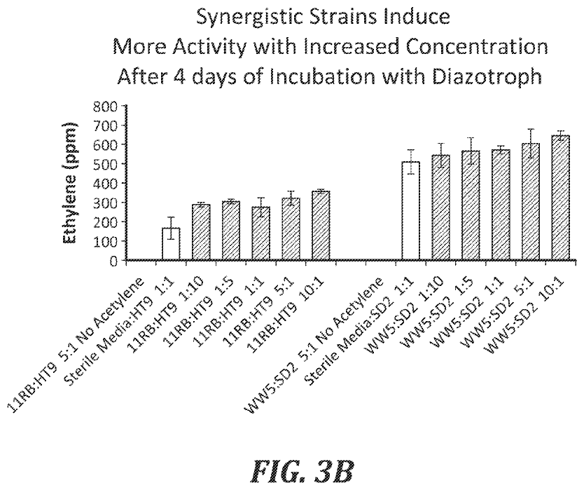

Synergistic strains induced more activity with increased concentration after 3 days incubation with diazotrophic strains. Acetylene reduction assay of diluted cultures in nitrogen free media (NFM) show that with increased ratios of synergistic strains to diazotrophs, nitrogen fixation activity increases as well. As illustrated in FIG. 3A, (1) incubating synergistic strains (striped bars) (e.g., HT1-2 and 11RB) for three days with diazotrophic strains (e.g., WP5 and SD2) and (2) increasing the ratio of the synergistic strain to the diazotrophic strain (white bars) (e.g., 5:1 and 10:1) resulted in an increase in synergistic nitrogen fixation.

Synergistic strains induced more activity with increased concentration after 4 days incubation with diazotroph. Acetylene reduction assay of diluted suspensions in NFM show that with increased ratios of synergistic strains to diazotrophs, nitrogen fixation activity increases as well. As illustrated in FIG. 3B, (1) incubating synergistic strains (striped bars) (e.g., 11RB and WW5) for four days with diazotrophic strains (white bars) (e.g., HT9 and SD2) and (2) increasing the ratio of the synergistic strain to the diazotrophic strain (e.g., 5:1 and 10.1) resulted in an increase in synergistic nitrogen fixation.

### Synergistic Activity is not a Common Trait for Bacteria Generally

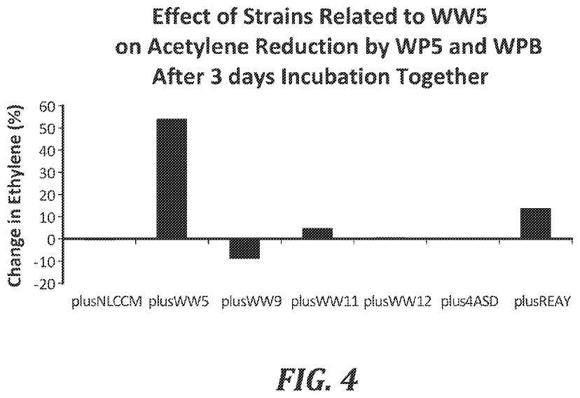

Effect of strains related to WW5 on acetylene reduction by WP5 and WPB after 3 days incubation together. Acetylene reduction assay performed with a mixture of diazotrophic strains that was treated as one cell suspension, each nitrogen fixer at OD₆₀₀ 0.2, which was mixed with a variety of strains related to WW5 as illustrated in FIG. 4. In FIG. 4, the variety of strains related to WW5 were first incubated with a mixture of diazotrophic strains for 3 days. As demonstrated in FIG. 3A, incubating synergistic strains with diazotrophic strains for at least 3 days increased nitrogen fixation. See FIGS. 3A and 3B. However, as illustrated in FIG. 4, incubation of the WW5 synergistic strain in the mixture of diazotrophic strains increased nitrogen fixation as expected, but this result was not observed for the variety of strains related to the WW5 synergistic strain. These results are important because these results demonstrate that synergistic activity seen in the disclosed partner strains is unique to these endophyte strains and is not a common trait of bacteria generally.

While illustrative embodiments have been illustrated and described, it will be appreciated that various changes can be made therein without departing from the spirit and scope of the invention.

1. A method to synergistically increase nitrogen acquisition in a plant in need thereof, the method comprising:
(i) generating an inoculant for a field treatment of the plant in need thereof, wherein the inoculant comprises a solution comprising an effective quantity of at least one live isolated endophyte strain, wherein the live isolated endophyte strain is isolated from one or more plants grown in a nutrient-limited and/or water-stressed environment; and
(ii) applying the inoculant to the plant in need thereof, where the at least one live isolated endophyte strain contacts at least one diazotrophic strain associated with the plant causing the diazotrophic strain to fix nitrogen at a higher rate compared to the nitrogen fixation rate of the diazotrophic strain in the absence of the at least one live isolated endophyte strain.

2-21. (canceled)

22. An inoculant to synergistically increase nitrogen acquisition in a plant in need thereof, the inoculant comprising an effective quantity of a solution derived from a lyophilized formulation comprising an effective amount of at least one live isolated endophyte strain from one or more plants grown in a nutrient-limited and/or water-stressed environment.

23. The inoculant of claim 22, wherein the inoculant is administered to a plant in need thereof, the at least one live isolated endophyte strain contacts at least one diazotrophic strain associated with the plant causing the diazotrophic strain to fix nitrogen at a higher rate compared to a nitrogen fixation rate of the diazotrophic strain in the absence of the at least one isolated endophyte strain.

24. The inoculant of claim 22, wherein the at least one live isolated endophyte strain comprises a 16S nucleic acid sequence as set forth in SEQ ID NOs: 1, 5, and 10.

25. The inoculant of claim 22, wherein the at least one live isolated endophyte strain comprises a 16S nucleic acid sequence as set forth in SEQ ID NO: 1.

26. The inoculant of claim 22, wherein the at least one live isolated endophyte strain comprises a 16S nucleic acid sequence as set forth in SEQ ID NO: 5.

27. The inoculant of claim 22, wherein the at least one live isolated endophyte strain comprises a 16S nucleic acid sequence as set forth in SEQ ID NO: 10.

28. The inoculant of claim 22, wherein the at least one live isolated endophyte strain comprises a marker comprising a sequence as set forth in Q SEQ ID NO:2, SEQ ID NO:3, SEQ ID NO:4, SEQ ID NO:6, SEQ ID NO:7, SEQ ID NO:8, SEQ ID NO:9, SEQ ID NO:11, SEQ ID NO:12, SEQ ID NO:13, SEQ ID NO:14, or any combination thereof.

29. The inoculant of claim 25, wherein the at least one live isolated endophyte strain comprises three markers comprising sequences as set forth in SEQ ID NOs: 2-4.

30. The inoculant of claim 26, wherein the at least one live isolated endophyte strain comprises four markers comprising sequences as set forth in SEQ ID NOs: 6-9.

31. The inoculant of claim 27, wherein the at least one live isolated endophyte strain comprises four markers comprising sequences as set forth in SEQ ID NOs: 11-14.

32. The inoculant of claim 29, wherein the at least one live isolated endophyte strain is of the species Sphingobium.

33. The inoculant of claim 30, wherein the at least one live isolated endophyte strain is of the species Herbiconiux.

34. The inoculant of claim 31, wherein the at least one live isolated endophyte strain is of the species Sphingobium.

35. The inoculant of claim 22, wherein the nutrient-limited and/or water-stressed environment is a primary substrate that comprises cobble or sand.

36. (canceled)

37. The inoculant of claim 22, wherein the nutrient-limited and/or water-stressed environment is a lava field, a desert, an arid environment, a semi-arid environment, a charred environment, or any combination thereof.

38. The inoculant of claim 22, wherein the plant in need thereof is a crop plant, a bioenergy crop plant, a forestry tree, a horticultural plant, a spice or medicinal plant, a turfgrass, or any combination thereof.

39-40. (canceled)

41. The inoculant of claim 22, wherein the solution derived from a lyophilized formulation further comprises at least one live isolated diazotrophic strain.

42. (canceled)

43. The inoculant of claim 41, wherein the ratio of the at least one live isolated endophyte strain to the at least one live isolated diazotrophic strain is 1+n:1, wherein n is an integer from 1 to 20.

44. A method to synergistically increase nitrogen fixation of at least one diazotrophic strain, the method comprising, contacting at least one diazotrophic strain with an effective quantity of a solution comprising an effective amount at least one live isolated endophyte strain as in claim 22, wherein the at least one live isolated endophyte is isolated from one or more plants grown in a nutrient-limited and/or water-stressed environment; and wherein contacting the live diazotrophic strain with the at least one live isolated endophyte strain causes the live diazotrophic strain to fix nitrogen at a higher rate compared to the nitrogen fixation rate of the live diazotrophic strain in the absence of the at least one live isolated endophyte strain.

45-53. (canceled)
